(19) United States
(12) Patent Application Publication
(10) Pub. No.: US 2006/0211967 A1
(43) Pub. Date: Sep. 21, 2006

(54) Knee orthosis

(76) Inventors: Ronan Reynolds, Long Beach, CA (US); Roger Weber, Pasadena, CA (US); Samuel Landsberger, Rancho Palos Verdes, CA (US)

Correspondence Address: Ronan Reynolds, PO Box 3953, Seal Beach, CA, 90740, US

(21) Appl. No.: 11/385,216

(22) Filed: Mar. 20, 2006

Related U.S. Application Data
(60) Provisional application No. 60/663,556, filed on Mar. 18, 2005.

(51) Int. Cl. A61F 5/00

(52) U.S. Cl. 602/23; 602/26

(57) ABSTRACT

A knee orthosis comprising of at least one deflection limiter (66) separated by gaps (10) on one or more struts (67), which are attached to a thigh shell (18) and a calf shell (68). The stiffness of the brace can easily be controlled in sagittal, transverse, and coronal planes whereby a person may obtain improved gait.

### CROSS-REFERENCE TO RELATED APPLICATION

This application claims the benefit of U.S. Provisional Application No. 60/663556 filed Mar. 18, 2005.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant No. H133E003001 awarded by the U.S. Department of Education.

### SEQUENCE LISTING OR PROGRAM

Not applicable

### BACKGROUND—FIELD OF THE INVENTION

The present invention relates to improvements in a knee orthosis (KO) used for supporting a person's knee along with extensions for supporting the user's hip and ankle. The orthosis of the present invention assists the user in extension and flexion of the user's hip, knee, and ankle while controlling sagittal, transverse, and/or coronal plane stiffnesses and deflections.

### BACKGROUND—DISCUSSION OF PRIOR ART

Knee Orthoses are currently used to support the knee when there is muscle weakness, bone deformity, joint deformity, joint damage, tendon damage, ligament damage, and/or whenever the loads and moments applied are higher than the person's physiology can handle.

Knee orthoses are commonly used in combination with ankle-foot orthoses to make knee-ankle foot orthoses (KAFO's), and hip orthoses to make Hip-knee-ankle-foot orthoses (HKAFO's).

The disadvantages of current KO's are that they cannot apply a moment across the knee that is biomechanically suitable. The human body uses its muscles to apply a non-linear moment across the knee for a full range of motion of the knee.

Current knee joints lock the knee entirely, lock the knee for all of stance phase of the gait cycle, or allow it to flex during stance phase, but not during swing phase. The normal human has high stiffness knee flexion during the load response part of stance phase, and low stiffness knee flexion during swing phase. No current knee orthoses provide these stiffness and range of motion properties.

The stiffness of an individual' knee varies with their weight, height, activity level, loads carried, strength and other factors. This brace allows an orthosis with a particular stiffness to be fabricated for a particular individual or class of individuals.

Current braces have very rigid flexion stops and/or extension stops, which cause sudden decelerations. These decelerations over time can damage a patient's knee and hip joints. Slower decelerations such as those applied by a normal person's muscles may be better.

U.S. patent application Ser. No. 11/111,973 by Reynolds et al shows a technology for controlling the ankle joint using segments on at least one strut.

There is a need for a knee orthosis, which applies a controlled non-linear stiffness across the knee in both flexion and extension that is similar to the stiffness applied by a normal person's muscles during gait. This stiffness needs to be reduced for sitting and swing phase.

### SUMMARY

The knee orthosis described in this application provides significantly enhanced performance compared to currently available knee orthoses. This orthoses has at least one strut, a thigh shell, a calf shell and at least one deflection limiter with gaps between the deflection limiter and the calf and thigh shell.

### DRAWINGS

FIG. 1 is a perspective right-side view of a right leg knee orthosis constructed in accordance with the invention. The knee orthosis used for the left leg is a mirror image.

FIG. 2 is a lateral side view of FIG. 1. It shows a thigh shell, defection limiters, and the spacers between the deflection limiters.

FIG. 3 is a view in detail of the portion indicated by the section line 3-3 in FIG. 2. It shows the spacers between the deflection limiters, and the holes for a knee extension assist device, and a locking/unlocking linkage.

FIG. 4 is a posterior perspective view of the knee orthosis with optional hip extension, optional Posterior Strut Ankle Foot Orthosis extension and optional Medial Lateral Strut Ankle Foot Orthosis extension. The orthoses used for the left leg are mirror images.

### NUMERALS IN DRAWINGS

7 Comfort Liner
10 Knee orthosis tab for attachment to ankle foot orthosis
11 Hole for Pin# 52
12 Knee hinge
13 Lock/Unlock Rod
14 Spacers
15 Extension assist
16 Thigh straps with hook and loop closure
17 Lock/unlock drive rod
18 Thigh shell
20 Hook and loop closure
22 Hook and loop closure
24 Hip joint
26 Pin to retain tab 62
27 Strut with slot for attachment to knee orthosis
28 Slot for attachment to Tab (10) on knee orthosis
30 Lock/unlock drive rod
32 Lock/unlock drive tube
34 Medial lateral strut AFO calf shell
36 AFO lateral support strut
38 Lock/unlock drive rod length adjustment
40 Lock/unlock drive rod length adjustment
42 Drive rod attachment to foot shell
44 Lateral ankle joint
46 Foot shell
48 Lock/unlock drive rod
50 Posterior strut AFO calf shell
52 Pin to hold knee orthosis tab in place
54 Rocker to change direction of drive rod
56 Lock/unlock drive rod connector
62 Tab for attachment to hip extension
63 Hole for Pin #26
64 Hook and loop closure
66 Deflection limiters
67 Strut
68 Calf shell
70 Hip band

### DESCRIPTION OF THE INVENTION

The present invention relates to a knee orthosis (“orthosis”) worn by a user on the user's leg or optionally on the user's hip and leg. The orthosis supports and assists users who have difficulty in standing and walking. Additionally, a user having no such difficulty can use the orthosis to assist their normal movement, which may assist the user's performance and endurance.

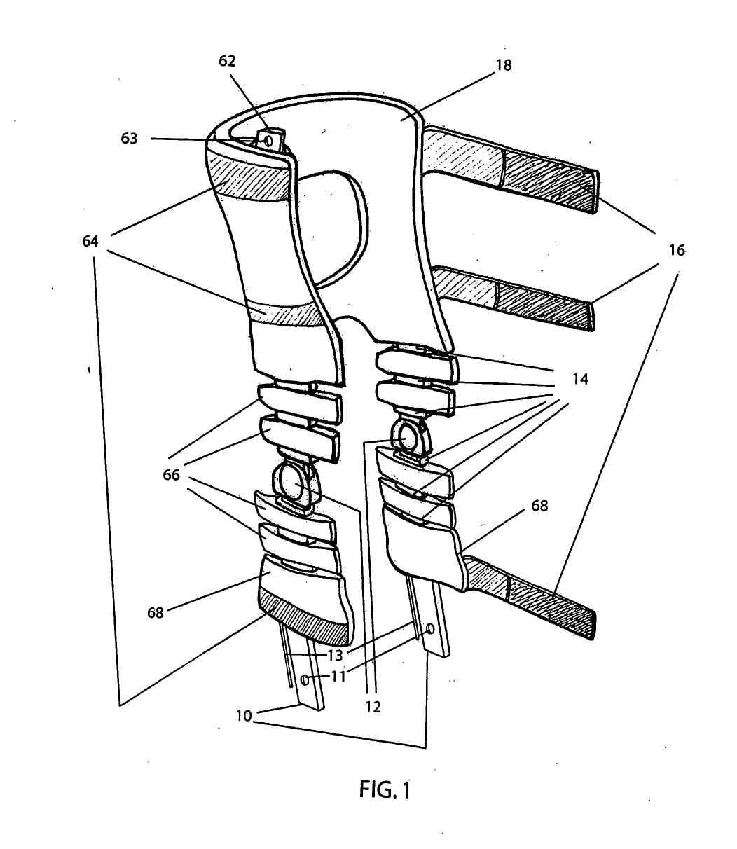

FIG. 1 shows a knee orthosis constructed and arranged in accordance with the invention. It consists of a thigh shell 18, which is securely attached to the wearer's thigh. It could be a generic shape or it may be custom made for a particular individual's leg. It could have anterior segments and/or posterior segments connecting the medial and lateral sides, and/or it could be spiral shaped and wrap around the thigh, and/or it could have straps with hook and loop closures and/or buckles or other attachments across the anterior sides 16, 4, and/or posterior straps, and/or encircling straps, and or no straps.

Attached to the thigh shell on the medial and lateral sides are struts 67(seen in the cross-section FIG. 3) which could consist of one piece or multiple struts. A single strut on only the medial or lateral side is also possible. The preferred embodiment has two struts on the medial and lateral side of the leg.

The orientation of the struts 67 from the thigh shell 18 to the knee joints 12 can be set at an angle to the line between the center of the wearer's anatomical hip joint to the center of the wearer's anatomical knee joint. This in combination with the below knee struts 67, and locked knee joints 12 allows the knee angle that the brace applies zero moment to the leg to be controlled. For example, if the knee joints 12 lock at 180 degrees and the above knee struts are oriented in 10 degrees of flexion to the hip to knee line, and the below knee struts 67 are oriented in 0 degrees of flexion to the knee to ankle line then the orthosis will apply zero moment at 10 degrees of knee flexion. This angle can be customized to the needs of the user.

Below the thigh shell, a one or more rigid segments called deflection limiters 66 surround the struts 67 or are attached to the struts 67. The deflection limiters 66 can be tapered, expanded, or parallel on the proximal and/or distal edges of the posterior and/or anterior sides to allow a different deflection or stiffness in flexion than in extension (tapering on the posterior side shown). Between the thigh shell 18 and the deflection limiters 66, and between the deflection limiters 66 themselves are rigid spacers 14 which allow the struts 67 to bend in a very controlled manner. The height of the spacers 14 and the shape of the deflection limiters 66 controls when the deflection limiters touch each other and the calf shells 68 and thigh shells 18 as the struts bend. These spacers could be separate pieces or incorporated into the shape of the deflection limiters 66. As strut 67 bends, the gaps controlled by the spacers 14 and the shape of the deflection limiters 66 progressively close. When the deflection limiters 66 touch each other or the thigh shell 18 or the calf shell 68, they effectively shorten the unsupported length of strut 67, increasing the stiffness of the knee orthosis.

Attached to the strut 67, below the deflection limiters 66, are the knee joints 12. This can be any type of knee joint such as a locking joint, a ratcheting lock joint, a stance-locking joint, range of motion-limited joint, and/or a free joint. The medial and lateral knee joints 12 may be of different types. In an additional configuration, both knee joints 12 can be removed allowing the strut to continue through this area and adding deflection limiters and spacers (if needed). In a further configuration, if only minimal support is needed, the medial knee joint 12, deflection limiters 66 and strut 67 may be removed entirely.

Below the knee joint 12 is another section of medial-lateral struts 67 with spacers 14 and deflection limiters 66 constructed in the same manner as above. The number of deflection limiters 66 and spacers 14 may vary as needed.

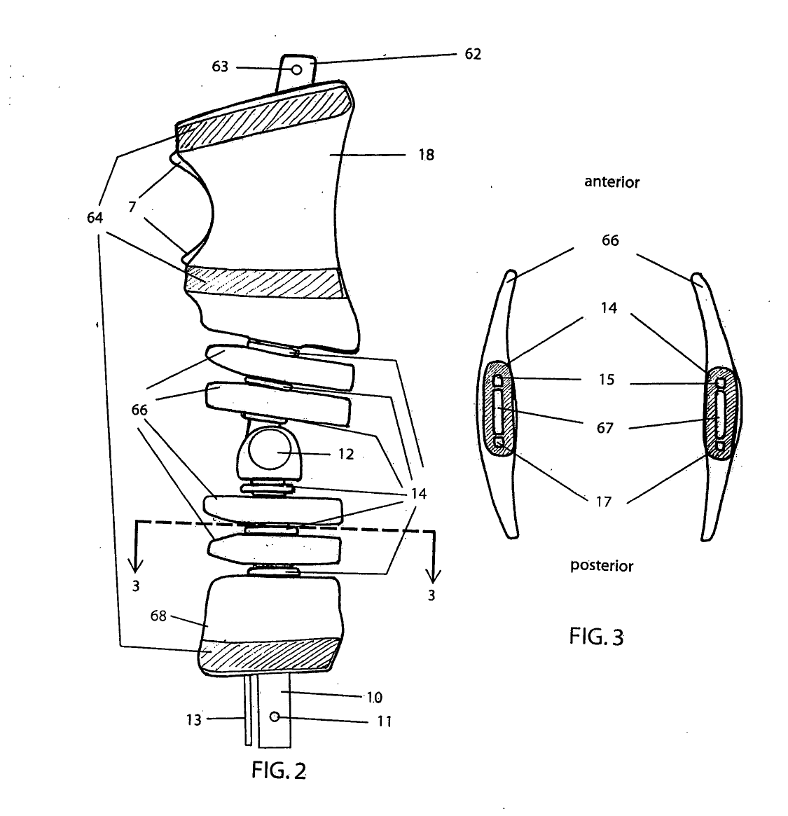

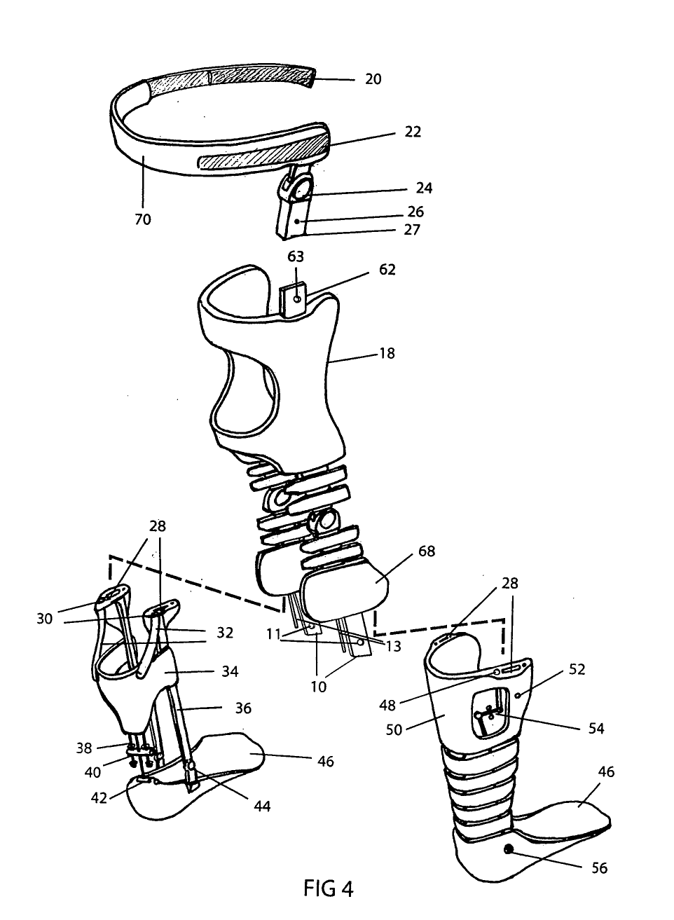

Towards its distal end, the strut 67 is attached to a calf shell 68. The calf shell 68 may be a generic shape or it may be custom made for a particular individual's leg. It could have anterior and/or posterior bars (not shown) connecting the medial and lateral sides, and/or it could be spiral shaped and wrap around the calf (not shown), and/or it could have straps with hook and loop closures 16, 4 and/or buckles-across the anterior side, and/or posterior straps, and/or encircling straps, and/or it can be constructed so that when it mates with an AFO, the AFO helps hold it on the lower leg (shown in FIG. 1, FIG. 2, FIG. 3, and FIG. 4).

The orientation of the struts 67 from the calf shell 68 to the knee joint 12 can be set at an angle to the line between the center of the wearer's anatomical knee joint and the center of the anatomical ankle joint. This in combination with the strut 67 above the knee joints 12 allows the knee angle that the brace applies zero moment across the knee to be controlled. For example, if the knee joints 12 lock at 180 degrees and the above knee struts 67 are oriented in 0 degrees of flexion to the hip to knee line, and the below knee struts 67 are oriented in 10 degrees of flexion to the knee to ankle line, then the orthosis will apply zero moment at 10 degrees of knee flexion. This angle can be customized to the needs of the user.

The gaps between the deflection limiters 66 themselves and between the calf shells 68 and thigh shells 18 and the deflection limiters 66 can be controlled by a variety of means such as through an integral step in the deflection limiters and the shells, through tapering or expanding the deflection limiters 66 or calf shells 68 and thigh shells 18 (shown in FIG. 2), and/or through the addition of rigid spacers 14 (as in FIG. 1, FIG. 2, FIG. 3., and FIG. 4), and by attaching the deflection limiters 66 directly to the strut 67.

FIG. 2 shows the lateral side view of the knee orthosis shown in FIG. 1 for use on the right leg. There is a comfort liner 63 on the inside of the thigh shell 18 and calf shell 68 to provide padding next to the wearer.

FIG. 3 shows the cross-section of the knee orthosis at section line 3-3. Visible are the struts 67 which each could consist of a single strut or multiple smaller struts, spacers 14, lock unlock rods 16 which could be round or square, and the holes 15 through which the extension assist mechanism passes. A cross-section through the struts 67 above the knee joints 12 would be similar.

Along the anterior side of each strut is an optional knee extension assist mechanism located in holes 15. This consists of a bungee cord, spring, flexible rod or combination of similar devices that is attached to the deflection limiters or the thigh and calf shells and crosses the knee joint on its anterior side. When the joint is unlocked and the knee bends the device resists flexion.

Along the posterior side of each strut is a lock/unlock rod 17 in a hole or channel. This can be used when a locking knee joint or stance control knee joint is used. This rod allows the knee brace to be unlocked during swing and locked during stance by the angle of the ankle (shown in FIG. 4) or by contact with the ground, or a foot switch, or a solenoid or other mechanism (not shown). If the direction of the lock/unlock rod 48, 17, 13 needs to be changed, a simple mechanism 54 such as a pivot can be used. The lock/unlock rod 13 can cross into the AFO adjacent to tab 10. It engages automatically when the AFO is attached. It may also be unlocked by full knee extension.

In an alternate configuration, some stance locking knee joints such as the Stance Phase Lock by Basko Healthcare do not require this lock/unlock rod 17. In an alternate configuration, a knee joint can be locked and unlocked by a lever or other mechanism that is triggered by knee flexion and weight bearing

In another embodiment, the length of the lock/unlock rod 48,13,17 and when it triggers can also be easily adjusted by a mechanism 38, 40 which consists of a bar attached to the footshell of the AFO by connector 42 which slides on the drive rod when the ankle is rotated. This bar which will only move the lock/unlock rod 13 when it contacts blocks attached to the drive rod. Other similar mechanisms such as those found on bicycle brakes will also work.

A stance locking knee joint can also be fabricated in this orthosis by using a freely rotating knee joint and a lock/unlock rod 17 made of a durable material such as metal. During plantarflexion of the ankle or some other locking signal, the rod pushes up and inserts into a slot or hole on the other side of the knee joint. This prevents rotation of the knee. If this hole is in a lock/unlock rod 17 that extends into the thigh shell, the knee joint 12 can be manually locked or unlocked by the wearer by lowering this lock/unlock rod 17 across the joint.

A separate or connected lock/unlock rod 17 can also extend from the knee joint into the thigh shell allowing the knee to be manually locked, unlocked, or placed into automatic stance control mode by the wearer or an assistant.

FIG. 4 is a posterior perspective view of the knee orthosis with optional hip extension, optional Posterior Strut Ankle Foot Orthosis extension and optional Medial Lateral Strut Ankle Foot Orthosis extension.

FIG. 4 also shows the optional pins bolts, rivets, ball snaps, pip pins or other connector 52, which attach the tabs 10, 62 to the AFO and hip orthosis. The shape of the knee orthosis calf shell 68 can be made with a step on the distal surface with a matching step on the proximal ankle foot orthosis calf shell 50 to facilitate connection and enhance support between the AFO and the knee orthosis.

At the bottom of the calf shell 88 are optional tabs 10 that allow the knee orthosis to be attached to an optional medial-laterally jointed AFO or posteriorly jointed AFO.

At the top of the thigh shell 18 is an optional tab 62 that allows the knee orthosis to attach to an optional hip orthosis.

The parts of a typical medial/laterally jointed AFO are the footshell 46, the ankle joint 44 which could be a free joint, a dorsiflexion assist joint, a dorsiflexion stop joint, a plantarflexion stop joint or a locked joint. Medial lateral struts 36 take the loads in the AFO. It has slots 28 for accepting tab 10. It has a lock/unlock actuator rod 30 in a tube 32 and an ankle foot orthosis calf shell 34.

The parts of a posterior strut AFO are a foot shell 46, an AFO calf shell 50, and a drive rod 48,13 attached to the footshell with a connector 56.

The parts of a typical hip orthosis are a hip band 70 a strap 20 with a buckle, connector or hook and loop closure 22. A hip joint 24 and a strut 27 with a slot to accept tab 62.

The same techniques used for adjusting flexion/extension stiffness described previously in this specification, such as adjusting the size of the spacers 14, the size or number of struts 67, or the shape of the deflection limiters 66, can be used to control medial and lateral knee stiffness and deflection in the transverse plane, as well as torsion in the coronal plane. This can be useful for compensating for knee and ankle varus/valgus.

The knee orthosis can be fabricated using various materials such as a fibrous material such as carbon fiber or fiberglass impregnated or pultruded with thermoset resin such as acrylic or epoxy, or out of metals such as aluminum or stainless steel. Additionally, it can be made using thermoplastic materials such as polyethylene and polypropylene.

### Preferred Embodiment

The preferred embodiment will depend on a particular patients needs. This knee orthosis provides a convenient selection of devices and characteristics to allow it to be tailored to allow a wide variety of patients the ability walk with an improved gait.

Many patients with severe calf and thigh weakness would use a configuration comprising of a posterior strut AFO connected to the knee orthosis. The knee orthosis strut 67 would be oriented 5 degrees forward of the line between the wearer's anatomical knee joint and hip joint, and 5 degrees forward of the line between the anatomical knee and ankle joints. This would make the knee moment equal to zero at a knee flexion angle of 10 degrees. There would be a knee extension assist consisting of elastic material. The knee joints 12 would be locking/unlocking knee joints with a lock/unlock rod 48,13 attached to the foot shell 46 of a posterior strut AFO connected through a pivot 54. The orthosis knee joint would be located near or slightly posterior to the anatomical knee joint.

### OPERATION OF THE INVENTION

The knee orthosis is attached to or around the lower leg of the wearer. First, the AFO is attached to the foot using straps if necessary. The shoe is then placed over the footshell 46. It is also possible to design the footshell 46 to fit over the shoe or not require a shoe at all.

The knee joints 12 are manually unlocked by moving the lock/unlock rod 17 inside the thigh shell 18 and the thigh shell 18 is placed around the wearers thigh and tab 10 is placed in slot 28. Straps 16 are then secured. If used, the hip orthosis is placed around the waist and tab 62 is attached to strut 27. Strap 20 is then secured. The wearer then extends their legs, manually locks the knee joints 12, stands up and walks. Sometimes additional assistive aids such as canes or walkers are also needed for ambulation. To remove the orthosis, the process is reversed.

In operation, almost any kind of knee stiffness curve can be predictably obtained using this invention. The size of the struts 67 determines the initial stiffness. The size of the spacers 14 and the shape of the deflection limiters 66 and calf shell 68 and thigh shells 18 determine how fast the stiffness increases. As the struts 67 bend, the gaps between the deflection limiters 66 progressively close and eventually touch each other or the calf shells 68 and thigh shells 18. This prevents further bending and the effective length of the struts 67 are shortened, increasing their stiffness. The width of the gaps is measured from the struts' 67 neutral axis and the gap height in an anterior/posterior manner determines how fast the stiffness increases. The smaller the gaps on the anterior side of the strut, the faster the stiffness in knee extension increases. The smaller the gaps on the posterior side, the faster the knee flexion stiffness increases.

The stiffer the extension assist material 15, the higher the extension assist force when the joint is unlocked.

The desired initial stiffness of the knee orthosis can be determined by dividing the normal knee moment by the normal knee angle at every point in the gait cycle. This desired value can be modified depending on the wearer's strength, and needs of the wearer. Standard beam bending equations applied to the struts 67 can be used to predict the initial stiffness of the knee orthosis in the sagittal plane. As each gap closes, the new stiffness can be calculated with the same equations by progressively shortening the effective length of the struts 67 by the height of deflection limiters 66 with their gaps closed. The maximum deflection can be calculated through basic geometrical formulas.

During gait, when the ankle plantarflexes under body weight, the knee is unstable and needs to be supported. When the ankle dorsiflexes, the ground reaction forces extend the knee joint so it does not need to be prevented from flexion. So a linkage across the ankle can be used to lock and unlock a knee joint 12.

In another embodiment, during gait, a ground reaction force corresponding to the weight of the wearer applied posterior to the ankle joint creates a flexion moment on the knee. So when a pushrod near the heel is compressed by body weight, it can trigger a lock at the knee. When the heel lifts up such as during push off, the spring-loaded pushrod returns to its original position and the knee is unlocked for swing through.

During the load response part of the gait cycle, a normal human flexes their knee. This brace allows the knee to be supportably flexed with a controlled stiffness during load response when the knee joint 12 is locked.

To sit down the wearer puts their knee into a neutral angle, pulls on the lock/unlock rod 17 in the thigh shell 18 to unlock the knee joint 12. Then the wearer sits down. The orthosis allows the knee to flex past 90 degrees. To stand up, the process is reversed.

### Description and Operation of Alternative Embodiments

An alternate configuration, to allow a variable stiffness curve, for a patient with a changing clinical picture, for changing terrain conditions, for more accurate initial stiffness setting, or many other reasons, consists of replacing some or all of the rigid spacers 14 with elastic spacers 14 made out of a material such as rubber and adding a device to compress the elastic spacers 14 such as a cam located medially or laterally to the strut (not shown) or a mechanism as simple as two locknuts on a threaded rod inserted into holes (not shown) fabricated in the deflection limiters 66 and the thigh shell 18 and calf shell 68. The holes would be located equidistant on the anterior and posterior sides of the strut. When the locknuts are turned in opposite directions on the threaded rod (or the cam rotated), the deflection limiters 66 and therefore the elastic spacers 14 are placed under compression, reducing the gaps between the deflection limiters 66 and between the thigh shells 18 and calf shells 68 thereby stiffening the knee orthosis. This configuration allows quick and simple brace stiffness modification.

Another alternative embodiment of this knee orthosis is where the deflection limiters 66 are two separate rectangular blocks attached solely to the anterior and posterior sides of the struts rather than a single piece that surrounds the strut as shown in FIG. 1. These rectangular blocks would work in the same as the previously described deflection limiters 66. The calf shell 50 and foot shell 52 would be similar to those previous described.

### CONCLUSION, RAMIFICATIONS, AND SCOPE OF INVENTION

Thus the reader will see that the knee orthosis which allows controlled non-linear knee stiffness in both flexion and extension comprising of deflection limiters 66 attached and/or surrounding a strut 67 which is attached to a thigh shell 18 and a calf shell 68, provides significant improvements in the ability to fit a particular patient's knee stiffness needs.

While principles of the invention are amenable to various modifications and alternative forms, specifics thereof have been shown by way of example in the drawings. It should be understood, however, that the intention is not to limit the invention to the particular embodiments described. On the contrary, the intention is to cover all modifications, equivalents, and alternatives falling within the spirit and scope of the disclosure and claims.

What is claimed is:

1. A knee orthosis comprising:
at least one strut member;
a thigh shell disposed at an upper end of the at least one strut member;
a calf shell disposed at the lower end of the at least one strut member;
a at least one deflection limiter disposed on the at least one strut member and between the thigh and calf shells; and
a plurality of gaps wherein a gap is formed between adjacent deflection limiters, a gap is formed between an uppermost deflection limiter and the thigh shell, and a gap is formed between a lowermost deflection limiter and the calf shell.

2. The orthosis according to claim 1, wherein each deflection limiter of the plurality of deflection limiters has a U-shaped cross-section or a substantially rectangular cross-section.

3. The orthosis according to claim 1, wherein each deflection limiter of the plurality of deflection limiters is either integrally formed about the at least one strut member or secured to the at least one strut member by fasteners.

4. The orthosis according to claim 1, wherein each deflection limiter of the plurality of deflection limiters includes a passageway extending therethrough, and wherein the passageway is configured to receive the at least one strut member therein.

5. The orthosis according to claim 1, further comprising:
A knee flexion stop; and
a knee extension stop.

6. The orthosis according to claim 5, wherein the flexion stop is defined by the posterior edges of the adjacent deflection limiters engaging each other.

7. The orthosis according to claim 6, wherein the at least one strut member further comprises an upper portion and a lower portion, and wherein a degree of rigidity of the flexion stop increases as the posterior edges of adjacent deflection limiters in the upper portion of the at least one strut member engage each other.

8. The orthosis according to claim 5, wherein the extension stop is defined by anterior edges of the adjacent deflection limiters engaging each other.

9. The orthosis according to claim 8, wherein the at least one strut member further comprises an upper portion and a lower portion, and wherein a degree of rigidity of the extension stop increases as the anterior edges of the adjacent deflection limiters in the upper portion of the at least one strut member engage each other.

10. The orthosis according to claim 1, wherein the at least one strut member further comprises an upper portion and a lower portion, and wherein the gaps formed in the lower portion of the at least one strut member are narrower relative to the gaps formed in the upper portion of the at least one strut member.

11. The orthosis according to claim 1, further comprising:
an adjustment mechanism for expanding or narrowing a height of each gap, wherein narrowing the height of each gap increases a rigidity of the orthosis, and wherein expanding the height of each gap decreases the rigidity of the orthosis.

12. The orthosis according to claim 11, wherein narrowing the height of each gap reduces a range of motion of the orthosis, and wherein expanding the height of each gap increases the range of motion of the orthosis.

13. The orthosis according to claim 11, wherein the adjustment mechanism is either a threaded rod and locknut combination or a twist draw mechanism.

14. The orthosis according to claim 1, further comprising:
at least one spacer disposed within each gap and which encompasses a corresponding portion of the at least one strut member,
wherein the spacer is compressed during extension and flexion as edges of the adjacent deflection limiters engage each other to define a stop, and
wherein the compressed spacer increases a rigidity of the orthosis.

15. The orthosis according to claim 14, further comprising:
an adjustment mechanism for expanding or narrowing a height of each gap to increase or decrease a rigidity of the orthosis.

16. The orthosis according to claim 15, wherein narrowing the height of each gap reduces a range of motion of the orthosis, and wherein expanding the height of each gap increases the range of motion of the orthosis.

17. The orthosis according to claim 1, wherein the orthosis further comprises at least one spacer integrally formed on a corresponding deflection limiter, and wherein each spacer includes at least one rounded edge that rollingly engages a neighboring deflection limiter.

18. The orthosis according to claim 1, further comprising:
a liner secured to at least one of an interior surface of the hip band, an interior surface of the thigh shell, interior surfaces of the plurality of deflection limiters, and an interior surface of the calf shell.

19. The orthosis according to claim 1, further comprising:
at least one strap for securing the knee orthosis to a user.

20. The orthosis according to claim 19, wherein the ankle-foot orthosis further includes an attachment mechanism for removably attaching the at least one strap to at least one of the hip band, the calf shell, the foot shell, and the plurality of deflection limiters.

21. The orthosis according to claim 20, wherein the attachment mechanism is one of a hook and loop fastener, a buckle, and a snap fastener.

22. The orthosis according to claim 1, further comprising:
at least one joint provided between at least one of a lowermost deflection limiter and the calf shell, the adjacent deflection limiters, and an uppermost deflection limiter and the thigh shell; and
at least one elastic member extending from the thigh shell to the calf shell to provide a knee flexion bias.

23. The orthosis according to claim 22, wherein a first end of the at least one elastic member is secured to the thigh shell, a second end of the at least one elastic member is secured to the calf shell, and a remaining portion of the at least one elastic member extends through aligned passageways provided in the thigh shell, the calf shell, and the plurality of deflection limiters.

24. A method of providing an orthosis comprising at least one strut member, a thigh shell, a calf shell, and a plurality of deflection limiters disposed between the thigh shell and calf shell with gradual stops in knee extension and knee flexion, the method comprising the steps of:
forming gaps in the orthosis between at least one of two adjacent deflection limiters, an uppermost deflection limiter and the thigh shell, and a lowermost deflection limiter and the calf shell, wherein the gaps formed in a lower portion of the at least one strut member are narrower than gaps formed in an upper portion of the at least one strut member; and
manipulating the orthosis in one of knee extension and knee flexion, wherein edges of adjacent deflection limiters forming the gaps in the lower portion of the at least one strut member engage before edges of adjacent deflection limiters forming the gaps in the upper portion of the at least one strut member engage each other.
